(19) United States
(12) Patent Application Publication
(10) Pub. No.: US 2024/0052295 A1
(43) Pub. Date: Feb. 15, 2024

(54) METHODS FOR CULTURING METHANOTROPHIC BACTERIA AND ISOLATING PROTEINS FROM BACTERIAL BIOMASS

(71) Applicant: Calysta, Inc., San Mateo, CA (US)

(72) Inventors: Yelena Stegantseva, San Mateo, CA (US); Lisa Marie Newman, San Jose, CA (US); Warren Kwan, San Mateo, CA (US); Chien-Seng Hwang, San Mateo, CA (US); Lorraine Joan Giver, Sunnyvale, CA (US); Celine Schiff-Deb, San Mateo, CA (US)

(21) Appl. No.: 17/766,625

(22) PCT Filed: Oct. 7, 2020
(86) PCT No.: PCT/US2020/054608

§ 371 Date: Apr. 5, 2022

Related U.S. Application Data
(60) Provisional application No. 62/911,747, filed on Oct. 7, 2019.

(51) Int. Cl. C12N 1/20; C07K 14/195

(52) U.S. Cl. CPC C12N 1/20; C07K 14/195

(57) ABSTRACT

The present disclosure provides method for generating biomass by culturing methanotrophic bacteria under low copper conditions. Also provided are generated biomass and protein isolate prepared from the biomass.

### BACKGROUND

### Technical Field

The present disclosure relates to bacterial culture, biomass and protein isolate generated from such culture. More specifically, the present disclosure relates to culturing a methanotrophic bacterium under low copper conditions to increase crude protein of bacterial biomass and protein isolate prepared from the biomass.

### Description of the Related Art

Protein production by conventional agriculture based food supply chains is becoming a major issue in terms of global environmental pollution and land and water scarcity. At the same time, the demand for high quality protein products such as those having high percentage crude protein is on the increase globally. Growing demand for protein cannot be met sustainably by increasing meat and dairy production because of the low efficiency of converting feed to meat and dairy products. Plant-based protein sources, such as beans, are nutritionally valuable sources of protein, but require arable land and water, both of which will become limiting. New solutions such as single cell protein (i.e., protein produced in microbial and algal cells) are being explored. Currently, microbial protein provides a relatively small proportion of human nutrition. In addition, production of microbial protein products may face issues related to high RNA content, bacteria-produced toxins, and immunogenicity.

### SUMMARY

The present disclosure provides methods for generating biomass from methanotrophic bacteria as well as the generated biomass and protein isolate prepared from the generated biomass.

In one aspect, the present disclosure provides a method for generating biomass, comprising: (a) continuously culturing a methanotrophic bacterium at a copper level no more than 100 mg copper per kg dry cell weight (DCW) to generate biomass.

In another aspect, the present disclosure provides a bacterial biomass comprising primarily, consisting essentially of, or consisting of, a biomass of a methanotrophic bacterium having a copper level no more than 100 mg copper per kg dry cell weight (DCW).

In a further aspect, the present disclosure provides a protein isolate prepared from a bacterial biomass that comprises primarily a biomass of a methanotrophic bacterium, wherein the protein isolate comprises at least 82% crude protein, preferably at least 85% crude protein.

### BRIEF DESCRIPTION OF THE SEVERAL VIEWS OF THE DRAWINGS

FIG. 1 is a flow chart showing an exemplary downstream processing of biomass used in Example 2.

FIG. 2 is a graph showing percentage crude protein of protein isolate prepared according to Example 2 from biomass ofMethylococcus capsulatus Bath cultured at a low copper level (25 mg copper/kg biomass) and under a normal copper level (154 mg copper/kg biomass). B091 and B093 are biomass from a continuous fermentation at the low copper level at different time points. B089 is biomass from a continuous fermentation at the normal copper level.

FIG. 3 is a graph showing crude protein (% of dry cell weight) of biomass of Methylococcus capsulatus Bath cultured at a low copper level (38 mg copper/kg biomass), a normal copper level (154 mg copper/kg biomass), and a high copper level (371 mg copper/kg biomass). The numbers #24 and #28 represent two separate fermentation runs.

FIG. 4 is a graph showing crude protein, fat and ash contents of biomass from Methylococcus capsulatus Bath grown at different copper levels (i.e., 23, 80, 96 and 140 mg copper/kg biomass).

### DETAILED DESCRIPTION

The present disclosure provides methods for culturing methanotrophic bacteria to generate biomass for preparing high quality protein products. It was discovered that methanotrophic bacteria cultured under low copper conditions produce biomass with higher crude protein, lower lipid, and/or lower ash contents than the bacteria cultured under normal or high copper conditions. Such biomass allows the production of protein isolate having high crude protein, increased yield, and/or minimal nucleic acid.

Without wishing to be bound to any theory, the present inventors hypothesize that the increase in crude protein of biomass from methanotrophic bacteria cultured under low copper conditions may be due to the reduction in the amount of the internal membrane structure (including lipid and membrane proteins) induced by low copper conditions.

In the present description, the term “about” means±10% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### Generating Biomass

In one aspect, the present disclosure provides a method for generating biomass by culturing a methanotrophic bacterium under low copper conditions.

1. Methanotrophic Bacterium

Methanotrophic bacteria have the ability to oxidize methane as a carbon and energy source. Methanotrophic bacteria are classified into three groups based on their carbon assimilation pathways and internal membrane structure: type I (gamma proteobacteria), type II (alpha proteobacteria, and type X (gamma proteobacteria).

Type I methanotrophs use the ribulose monophosphate (RuMP) pathway for carbon assimilation whereas type II methanotrophs use the serine pathway. Type X methanotrophs use the RuMP pathway but also express low levels of enzymes of the serine pathway.

Methanotrophic bacteria include obligate methanotrophs, which can only utilize C₁ substrates for carbon and energy sources, and facultative methanotrophs, which naturally have the ability to utilize some multi-carbon substrates as a carbon and energy source.

Exemplary facultative methanotrophs include some species of Methylocella, Methylocystis, and Methylocapsa (e.g Methylocella silvestris, Methylocella palustris, Methylocella tundrae, Methylocystis daltona strain SB2, Methylocystis bryophila, and Methylocapsa aurea KYG), Methylobacterium organophilum (ATCC 27,886), Methylibium petrolelphilum, or high growth variants thereof.

Exemplary obligate methanotrophic bacteria include Methylococcus capsulatus Bath (NCIMB 11132), Methylomonas sp. 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus Y (NRRL B-11,201), Methylomonas flagellata sp. AJ-3670 (FERM P-2400), Methylacidiphilum infernorum and Methylomicrobium alcallphilum, or high growth variants thereof.

In certain embodiments, the methanotrophic bacterium is a methanotrophic bacterium that expresses soluble methane monooxygenase (sMMO). MMOs catalyze oxidation of methane to methanol in methanotrophic bacteria.

Preferably, the methanotrophic bacterium is Methylococcus, Methylocystis, Methylosinus, or Methylocella, including those that express sMMO.

In certain embodiments, the methanotrophic bacterium is Methylococcus capsulatus, including Methylococcus capsulatus Bath, Methylococcus capsulatus Texas, and Methylococcus capsulatus Aberdeen. Preferably, the methanotropic bacterium is Methylococcus capsulatus Bath. It is a thermophilic bacterium with an optimum growth temperature at about 45° C. M. capsulatus Bath is a Type I methanotroph.

2. Low, Normal and High Copper Conditions

The term “low copper conditions” refers to continuous culture conditions where the amount (or level) of copper in a continuous culture is at most 100 mg copper (i.e., elemental copper or copper element) per kg dry cell weight (DCW).

“Continuous culture conditions” refers to conditions under which a methanotrophic bacterium is cultured in a continuous culturing system wherein a defined culture medium (or its component(s)) is continuously added to the system while an equal amount of used medium is removed simultaneously for processing.

“Continuous culture” refers to the mixture of a culture medium and bacteria cultured in the medium under continuous culture conditions.

“Dry cell weight (DCW)” refers to the dry weight of biomass harvested from a bacterial culture.

A specified amount of copper element is typically provided by a corresponding or equivalent amount of a copper salt that contains the same number of mole of copper element. For example, 100 mg copper is about 1.57 mmol, and may be provided by about 394 mg CuSO₄.5H₂O.

The term “normal copper conditions” refers to continuous culture conditions where the amount of copper in a continuous culture is in the range of 100 mg to 200 mg copper per kg dry cell weight (DCW).

The term “high copper conditions” refers to continuous culture conditions where the amount of copper in a continuous culture is more than 200 mg copper per kg dry cell weight (DCW).

Specified copper conditions are typically set up by controlling Cu feed rates in view of DCW harvest rates. For example, for a low copper (Cu) concentration of 50 μg Cu/g of DCW (dry cell weight) and harvest rate 5 g/L/h of DCW, Cu (such as provided byCuSO₄.5H₂O) feed should be 250 μg Cu/L/h.

In certain embodiments, copper concentrations may be controlled by the use of a device (e.g., a pump) to feed a continuous culture at a defined rate.

In certain embodiments, the copper level under low copper conditions is from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, preferably from 20 to 80, from 20 to 70, from 20 to 60, from 20 to 50, or from 20 to 40 mg copper/kg biomass.

In certain embodiments, the copper level under normal copper conditions is from 100 to 180, from 100 to 170, from 100 to 160, from 100 to 150, from 100 to 140, from 100 to 130 mg copper/kg biomass.

In certain embodiments, the copper level under high copper conditions is from 200 to 800, from 200 to 700, from 200 to 600, from 200 to 500, or from 200 to 400 mg copper/kg biomass.

3. Culturing Methanotrophic Bacterium

Methanotrophic bacteria may be grown by continuous culture methodologies in a controlled culture unit, such as a fermenter, bioreactor, hollow fiber cell, or the like. Continuous culture systems are systems where a defined culture medium (or its component(s)) is continuously added to a controlled culture unit while an equal amount of used (“conditioned”) media is removed simultaneously for processing. Continuous culture systems generally maintain the cells at a constant high, liquid phase density where cells are primarily in logarithmic growth phase.

A continuous culture system allows for the modulation of one or more factors that affect cell growth or end product concentration. For example, one method may maintain a limited nutrient at a fixed rate (e.g., carbon source, nitrogen) and allow one or more other parameters to change over time. In certain embodiments, several factors affecting growth may be continuously altered while cell concentration, as measured by media turbidity, is kept constant. The goal of a continuous culture system is to maintain steady state growth conditions while balancing cell loss due to media being drawn off against the cell growth rate. Methods of modulating nutrients and growth factors for continuous culture processes and techniques for maximizing the rate of product formation are well known in the art (see, e.g., Thomas D. Brock, Biotechnology: A Textbook of Industrial Microbiology, 2^{nd} Ed. (1989) Sinauer Associates, Inc., Sunderland, MA; Deshpande, Appl. Biochem. Biotechnol. 36:227, 1992).

In certain embodiments, culture medium includes a carbon substrate as a source of energy for a methanotrophic bacterium. Suitable substrates include Ci substrates, such as methane, methanol, formaldehyde, formic acid (formate), carbon monoxide, carbon dioxide, methylated amines (methylamine, dimethylamine, trimethylamine, etc.), methylated thiols, or methyl halogens (bromomethane, chloromethane, iodomethane, dichloromethane, etc.). In certain embodiments, culture media may comprise a single Ci substrate as the sole carbon source for a methanotrophic bacterium, or may comprise a mixture of two or more Ci substrates (mixed Ci substrate composition) as multiple carbon sources for a methanotrophic bacterium. In certain embodiment, natural gas (which primarily contains methane) may be used as a carbon source.

During bacterial culture, the pH of the fermentation mixtures will generally be regulated to be between about 6 and about 8, such as between about 6 and about 7, between about 7 and about 8, or between about 6.5 and 7.5.

During bacterial culture, the temperature is maintained to be in the range optimal for the cultured bacterium. For example, for M. capsulatus Bath, the temperature may be between 40° C. and 45° C.

Preferably, the methanotrophic bacterium is M. capsulatus Bath. M. capsulatus Bath may be cultured using methane as its carbon source, air or pure oxygen for oxygenation, and ammonia as the nitrogen source. In certain embodiments, a carbon feedstock comprising methane used for culturing M. capsulatus is natural gas or unconventional natural gas. In addition to these substrates, the bacterial culture will typically require water, phosphate, and several minerals such as magnesium, calcium, potassium, irons, copper, zinc, manganese, nickel, cobalt and molybdenum. Exemplary culture media include Higgins minimal nitrate salts medium (NSM) or MM-W1 medium, master mix feed (MMF) as described in Example 1, medium MMF1.1, medium MMS1.0, or AMS medium. The copper concentrations of these media may be adjusted as described above. Exemplary culturing conditions with different copper concentrations are provided in Example 1.

The composition of medium MMS 1.0 is as follows: 0.8 mM MgSO₄·7H₂O, 30 mM NaNO₃, 0.14 mM CaCl₂, 1.2 mM NaHCO₃, 2.35 mM KH₂PO₄, 3.4 mM K₂HPO₄, 20.7 μM Na₂MoO₄·2H₂O, 6 μM CuSO₄·5H₂O, 10 μM Fe^{III}—Na-EDTA, and 1 mL per liter of a trace metals solution (containing per liter: 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA). The final pH of the media is 7.0±0.1.

The AMS medium contains the following per liter: 10 mg NH₃, 75 mg H₃PO₄·2H₂O, 380 mg MgSO₄·7H₂O, 100 mg CaCl₂·2H₂O, 200 mg K₂SO₄, 75 mg FeSO₄·7H₂O, 1.0 mg CuSO₄·5H₂O, 0.96 mg ZnSO₄·7H₂O, 120 μg CoCl₂·6H₂O, 48 μg MnCl₂·4H₂O, 36 μg H₃BO₃, 24 μg NiCl₂·6H₂O and 1.20 μg NaMoO₄·2H₂O.

The composition of medium MMF1.1 is as follows: 0.8 mM MgSO₄·7H₂O, 40 mM NaNO₃, 0.14 mM CaCl₂, 6 mM NaHCO₃, 4.7 mM KH₂PO₄, 6.8 mM K₂HPO₄, 20.7 μM Na₂MoO₄·2H₂O, 6 μM CuSO₄·5H₂O, 10 μM Fe^{III}—Na-EDTA, and 1 mL per liter of trace metals solution (containing, per liter 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA).

Suitable fermenters for culturing methanotrophic bacteria may be of the loop-type or air-lift reactors. Exemplary fermenters include U-loop fermenters (see U.S. Pat. No. 7,579,163, WO2017/218978), serpentine fermenters (see WO 2018/132379), and Kylindros fermenters (see WO 2019/0366372).

In certain embodiments, the methanotrophic bacterium is cultured under good manufacturing practice (GMP) conditions. As used herein, the term “good manufacturing practice” or “GMP” refers to regulations promulgated by the US Food and Drug Administration under the authority of the Federal Food, Drug, and Cosmetic Act in 21 CFR 110 (for human food) and 111 (for dietary supplements) or comparable regulations set forth in jurisdictions outside the U.S that describe conditions and practices that are necessary for the manufacturing, processing, packing or storage of food to ensure its safety and wholesomeness.

In certain embodiments, the methanotrophic bacterium is cultured as an isolated culture without the presence of another organism. In certain other embodiments, the methanotrophic bacterium may be grown with one or more heterologous organisms (e.g., one or more heterologous bacteria) that may aid with growth of the methanotrophic bacterium. For example, a methanotrophic bacterium (e.g., Methylococcous capsulatus Bath) may be cultured with Cupriavidus sp., Anuerinibacillus danicus, or both and optionally in combination with Brevibacillus agri.

4. Bacterial Biomass

The term “bacterial biomass” refers to organic material collected from bacterial culture. Bacterial biomass primarily (i.e., more than 50% w/w) comprises bacterial cells, but may include other materials such as lysed bacterial cells, bacterial cell membranes, inclusion bodies, and extracellular material (e.g., products secreted or excreted into the culture medium), or any combination thereof that are collected from bacterial fermentation along with bacterial cells. Preferably, the biomass includes more than 60%, 70%, 75%, 80%, 85%, 90% or 95% cells collected from bacterial fermentation.

Bacterial biomass may be harvested from bacterial culture by various techniques, such as sedimentation, microfiltration, ultrafiltration, spray drying. Preferably, biomass is harvested from bacterial culture by centrifugation (e.g., at 4,000×g for 10 minutes at 10° C. For example, a fermentation broth (cells and liquid) may be collected and centrifuged. After centrifugation, the liquid can be discarded, and the precipitated cells may be saved and optionally lyophilized.

In certain embodiments, bacterial biomass consists essentially of or consists of the biomass harvested from a methanotrophic bacterium cultured under low copper conditions has a copper level no more than 100 mg copper per kg DCW (mg/kg). In certain embodiments, the bacterial biomass and/or the biomass of methanotrophic bacterium has a copper level from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, or from 20 to 40 mg copper/kg DCW.

In certain embodiments, the bacterial biomass and/or the biomass of methanotrophic bacterium cultured under low copper conditions has at least 71% crude protein, such as at least 72%, 73%, 74%, 75%, 76%, 77%, 78%, 79%, 80%, or 81% crude protein. “Crude protein,” “crude protein content,” “crude protein concentration,” or “percentage crude protein” is a measurement of nitrogen in a protein sample. The amount of nitrogen is indicative of the amount of protein in the sample. The crude protein content of biomass or protein isolate disclosed herein is measured by the Dumas method. In certain embodiments, the bacterial biomass and/or the biomass of methanotrophic bacterium is composed of about 71% to about 99%, about 75% to about 99%, about 80% to about 99%, 82% to about 99%, about 71% to about 95%, about 75% to about 95%, about 80% to about 95%, about 82% to about 95%, about 71% to about 90%, about 75% to about 90%, about 80% to about 90%, about 82% to about 90%, about 71% to about 85%, about 75% to about 85% crude protein.

In certain embodiments, the bacterial biomass and/or the biomass of methanotrophic bacterium cultured under low copper conditions has at least 60%, 61%, 62%, 63%, 64%, 65%, 66%, 67%, 68%, 69%, 70%, 71%, 72%, 73%, 74%, 75%, 76%, 77%, 78%, 79%, or 80% true protein. “True protein,” “true protein content,” “true protein concentration,” or “percentage true protein” is a measurement of crude protein minus the non-protein nitrogen content in a protein sample. In certain embodiments, the bacterial biomass and/or the biomass of methanotrophic bacterium is composed of about 60% to about 99%, about 65% to about 99%, about 70% to about 99%, about 75% to about 99%, about 80% to about 99%, about 60% to about 95%, about 65% to about 95%, about 70% to about 95%, about 75% to about 95%, about 80% to about 95%, about 60% to about 90%, about 65% to about 90%, about 70% to about 90%, about 75% to about 90%, about 80% to about 90%, about 60% to about 85%, about 65% to about 85%, about 70% to about 85%, about 75% to about 85%, about 60% to about 80%, about 65% to about 80%, about 70% to about 80%, about 60% to about 75%, or about 60% to about 70% true protein. In certain embodiments, the bacterial biomass and/or the biomass of methanotrophic bacterium cultured under low copper conditions has at most 14% such as at most 13%, 12%, or 11% ash. “Ash” is material left over in a sample that is burned (e.g., in furnace for 12-18 hours or overnight at 550° C.).

In certain embodiments, the bacterial biomass and/or the biomass of methanotrophic bacterium cultured under low copper conditions has at most 10%, such as at most 9%, 8%, 7%, 6%, or 5% nucleic acid. The nucleic acid content of biomass or protein isolate disclosed herein is measured using a Lucigen Masterpure Complete DNA & RNA Purification Kit MC85200.

In certain embodiments, the bacterial biomass and/or the biomass of methanotrophic bacterium cultured under low copper conditions has at most 10%, 9%, 8%, 7.5%, 7%, 6%, or 5% crude fat. Crude fat may be measured by acid hydrolysis followed by organic solvent extraction. Briefly, fats or lipids in the bacterial biomass and/or the biomass of methanotrophic bacterium are first broken down via acid hydrolysis before being extracted via a solvent (e.g., ether or hexane). The solvent is then evaporated, and the material that remains is referred to “crude fat.”

In certain embodiments where a methanotrophic bacterium is cultured with one or more heterologous organisms, such as Methylococcous capsulatus Bath cultured with Cupriavidus sp., Anuerinibacillus danicus or both and optionally in combination with Brevibacillus agri, the bacterial biomass may comprise biomass from the heterologous organism(s) in addition to biomass from the methanotrophic bacterium.

Preferably, the bacterial biomass comprises primarily (i.e., more than 50%, such as more than 55%, more than 60%, more than 65%, more than 70%, more than 75%, more than 80%, more than 85% or more than 90% by weight) biomass from the methanotrophic bacterium.

In certain embodiments where a methanotrophic bacterium is cultured with one or more heterologous organisms, the bacterial biomass and/or the biomass of the methanotrophic bacterium has a copper level no more than 100 mg copper per kg DCW (mg/kg). In certain embodiments, the bacterial biomass and/or the biomass of the methanotrophic bacterium has a copper level from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, or from 20 to 40 mg copper/kg DCW.

In certain embodiments where a methanotrophic bacterium is cultured with one or more heterologous organisms, the bacterial biomass and/or the biomass of the methanotrophic bacterium has at least 71%, 72%, 73%, 74%, 75%, 76%, 77%, 78%, 79%, 80%, or 81% crude protein, such as about 71% to about 99%, about 75% to about 99%, about 80% to about 99%, 82% to about 99%, about 71% to about 95%, about 75% to about 95%, about 80% to about 95%, about 82% to about 95%, about 71% to about 90%, about 75% to about 90%, about 80% to about 90%, about 82% to about 90%, about 71% to about 85%, about 75% to about 85% crude protein.

In certain embodiments where a methanotrophic bacterium is cultured with one or more heterologous organisms, the bacterial biomass and/or the biomass of methanotrophic bacterium cultured under low copper conditions has at least 60%, 61%, 62%, 63%, 64%, 65%, 66%, 67%, 68%, 69%, 70%, 71%, 72%, 73%, 74%, 75%, 76%, 77%, 78%, 79%, or 80% true protein, such as about 60% to about 99%, about 65% to about 99%, about 70% to about 99%, about 75% to about 99%, about 80% to about 99%, about 60% to about 95%, about 65% to about 95%, about 70% to about 95%, about 75% to about 95%, about 80% to about 95%, about 60% to about 90%, about 65% to about 90%, about 70% to about 90%, about 75% to about 90%, about 80% to about 90%, about 60% to about 85%, about 65% to about 85%, about 70% to about 85%, about 75% to about 85%, about 60% to about 80%, about 65% to about 80%, about 70% to about 80%, about 60% to about 75%, or about 60% to about 70% true protein.

In certain embodiments where a methanotrophic bacterium is cultured with one or more heterologous organisms, the bacterial biomass and/or the biomass of the methanotrophic bacterium has at most 14% such as at most 13%, 12%, or 11% ash.

In certain embodiments where a methanotrophic bacterium is cultured with one or more heterologous organisms, the bacterial biomass and/or the biomass of the methanotrophic bacterium has at most 10%, such as at most 9%, 8%, 7%, 7.5%, 6%, or 5% nucleic acid.

In certain embodiments where a methanotrophic bacterium is cultured with one or more heterologous organisms, the bacterial biomass and/or the biomass of methanotrophic bacterium cultured under low copper conditions has at most 10%, 9%, 8%, 7%, 6%, or 5% crude fat.

In certain embodiments, the biomass is harvested from a methanotrophic bacterium cultured under GMP conditions.

### Preparing Protein Isolate

In a related aspect, the present disclosure provides a method for generating protein isolate by purifying proteins from biomass obtained as disclosed herein.

The term “protein isolate” refers to a composition that comprises primarily proteins isolated, extracted or purified from a bacterial biomass that comprises primarily, consisting essentially of, or consisting of a biomass of a methanotrophic bacterium. A composition that comprises primarily proteins isolated from a bacterial biomass refers to a composition of which more than 50% (e.g., more than 55%, 60%, 70%, 75% or 80%) by weight is proteins from the bacterial biomass. Through the use of protein isolation, extraction or purification technique(s), protein isolate has higher protein content (e.g., measured by percentage crude protein) than the bacterial biomass from which the protein isolate is prepared. However, protein isolation, extraction or purification does not need to be to the extent that individual proteins are separated from each other. Instead, protein isolate in general comprises a mixture of proteins isolated, extracted or purified from a bacterial biomass in which at least some of the other components (e.g., nucleic acids or lipids) in the bacterial biomass are removed.

In general, bacterial biomass harvested from a culture of a methanotrophic bacterium goes through a cell disruption step (e.g., homogenization, beadmilling, freeze/thaw cycles, enzymatic digestion, sonication, French press, and chemical solubilization) to generate a lysate first followed by protein separation and/or concentration step(s) (e.g., flocculation, microfiltration, ultrafiltration, nanofiltration, precipitation, isoelectric precipitation (via pH or salts), solvent precipitation, chromatographic methods based on adsorption, ion exchange chromatography, size exclusion chromatography, or affinity, and heat denaturation) of the lysate to generate protein isolate. The resulting protein isolate may be liquid protein isolate or further dried (e.g., via spray drying, lyophilization, evaporation, vacuum drying) to obtain dry protein isolate.

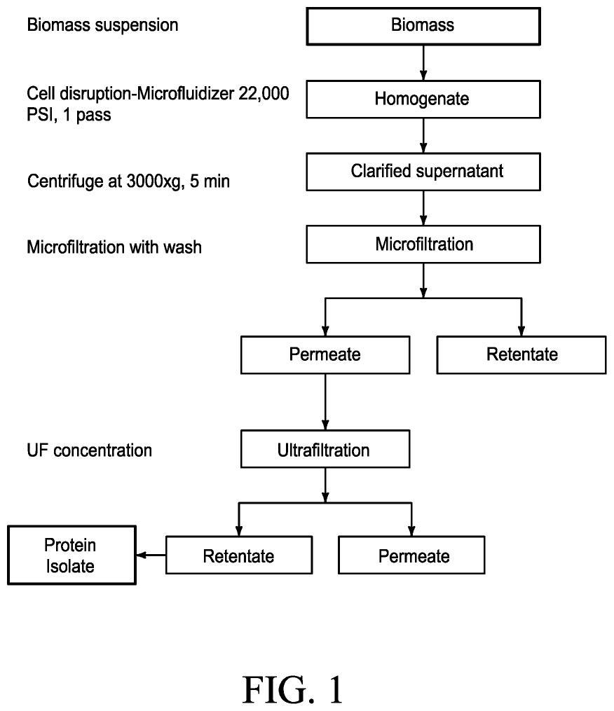

An exemplary workflow of preparing protein isolate is to homogenize bacterial biomass (e.g., via a microfluidizer), centrifuge the homogenate to obtain clarified supernatant, subject the supernatant to microfiltration, subject the resulting permeate to ultrafiltration, and lyophilize the resulting retentate to obtain protein isolate as a dry powder. A schematic representation of a specific example of the workflow used in Example 1 is shown in FIG. 1.

Another exemplary workflow of preparing protein isolate is to homogenize bacterial biomass (e.g., via a microfluidizer), add a flocculant to the homogenate, centrifuge to remove cell debris and obtain clarified supernatant, subject the clarified supernatant to ultrafiltration, and lyophilize the resulting retentate to obtain protein isolate as a dry powder.

A further exemplary workflow of preparing protein isolate is to homogenize bacterial biomass (e.g., via a microfluidizer), add a flocculant to the homogenate, centrifuge to remove nucleic acids and/or cell debris and obtain clarified supernatant, subject the clarified supernatant to acid precipitation (e.g., adjusting pH with H₂SO₄ to about 4.5), optionally wash precipitated proteins, neutralize and re-suspend precipitated proteins (e.g., adjust pH with sodium hydroxide to 7), and lyophilze re-suspended proteins to obtain protein isolate as a dry powder.

Another exemplary workflow of preparing protein isolate is to homogenize bacterial biomass (e.g., via a microfluidizer), centrifuge the homogenate to remove cell debris and obtain clarified supernatant, subject the clarified supernatant to acid precipitation (e.g., adjusting pH with H₂SO₄ to about 4.5), optionally wash precipitated proteins, neutralize and re-suspend precipitated proteins (e.g., adjust pH with sodium hydroxide to 7), and lyophilze re-suspended proteins to obtain protein isolate as a dry powder.

Flocculants, especially cationic flocculants, that may be used in preparing protein isolate to reduce nucleic acid and/or cell debris include chitosan (e.g., chitosan derived from shellfish or fungal source), poly-L-lysine, polyethylenimine (PEI), DEAE (diethylaminoethyl ion exchange resin), DEAE-dextran hydrochloride, amidated pectin (e.g., amidated low methoxyl pectin), Tramfloc 860 series (alkylamine epichlorohydrin), pDADMACs (diallydimethylammonium chloride), Isinglass, gelatin, egg white. Preferably, a flocculant is chitaosan, poly-L-lysine, DEAE, alkylamine epichlorhydrins, and pDADMACs.

Additional description about preparing protein isolate from bacterial biomass may be found in the U.S. provisional application titled “Food Compositions Comprising Methylococcus Capsulatus Protein Isolate” filed on Oct. 7, 2019.

In certain embodiments, culturing methanotrophic bacteria under low copper conditions may increase the yield of protein isolate compared to culturing the methanotrophic bacterium under normal or high copper conditions. In some embodiments, the ratio of the yield of protein isolate prepared from biomass of a methanotrophic bacterium cultured under low copper conditions to the yield of protein isolate prepared from biomass of the methanotrophic bacterium cultured under normal or high copper conditions (e.g., at the copper level of 150 mg copper per kg DCW) is at least 1.2, 1.3, 1.4, 1.5, 1.6, 1.7, 1.8, 1.9, or preferably at least 2.0, 2.1, 2.2, 2.3, 2.4, or 2.5.

The “yield of protein isolate” refers to the percentage of protein from biomass homogenate retained in protein isolate. In other words, the yield of protein isolate is the percentage of protein of protein isolate when setting the biomass homogenate from which the protein isolate is prepared to be 100%. Biomass homogenate is a mixture resulted from homogenizing biomass (see e.g., FIG. 1). The protein content may be measured by the BCA method (Smith et al., Anal Biochem. 150(1):76-85, 1985), such as using ThermoFisher Scientific Pierce BCA Protein Assay Kit).

In certain embodiments, the yield of the protein isolate is at least 10%, such as at least 11%, 12%, 13%, 14%, 15%, 16%, 17%, 18%, 19%, preferably at least 20%, 21%, 22%, 23%, 24% or 25%.

Protein isolate prepared from a methanotropic bacterium cultured under low copper conditions typically has higher crude protein content than protein isolate prepared in the same manner from the methanotropic bacterium cultured under normal or high copper conditions. In certain embodiments, the protein isolate prepared from a methanotropic bacterium cultured under low copper conditions has at least 82%, such as at least 83%, at least 84%, at least 85%, at least 86%, at least 87%, at least 88%, at least 89%, at least 90%, at least 91%, at least 92%, at least 93%, at least 94%, at least 95%, or at least 96% crude protein. In certain embodiments, the protein isolate is composed of about 82% to about 99%, about 85% to about 99%, about 90% to about 99%, about 82% to about 95%, about 85% to about 95%, or about 90% to about 95% crude protein.

In certain embodiments, the protein isolate is composed of at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% true protein. In certain embodiments, the Methylococcus capsulatus protein isolate is composed of about 65% to about 99%, about 65% to about 95%, about 65% to about 90%, about 65% to about 85%, about 65% to about 80%, about 65% to about 75%, about 70% to about 99%, about 70% to about 95%, about 70% to about 90%, about 70% to about 85%, about 70% to about 80%, about 75% to about 99%, about 75% to about 95%, about 75% to about 90%, about 75% to about 85%, about 80% to about 99%, about 80% to about 95%, or about 80% to about 90% by weight true protein. Protein isolate prepared from a methanotropic bacterium cultured under low copper conditions preferably contains minimal amount of nucleic acid to minimize potential advance effects of a high nucleic acid level to animals or human that consume the protein isolate (e.g., causing gout and kidney stones). In certain embodiments, the protein isolate prepared from a methanotropic bacterium cultured under low copper conditions has at most 10%, 9%, 8%, 7%, 6%, preferably, at most 5%, 4%, 3%, 2% or 1% nucleic acid.

Protein isolate prepared from a methanotropic bacterium cultured under low copper conditions preferably contains a minimal amount of ash. In certain embodiments, Methylococcus capsulatus protein isolate has less than about 10%, 9%, 8%, 7%, 6%, 5%, 4%, 3%, 2%, or 1% by weight ash.

Protein isolate prepared from a methanotropic bacterium cultured under low copper conditions preferably contains a minimal amount of fat. In certain embodiments, Methylococcus capsulatus protein isolate has less than about 10%, 9%, 8%, 7%, 6%, 5%, 4%, 3%, 2%, or 1% by weight crude fat.

In certain embodiments, the protein isolate has a copper level at most 100, 90, 80, 70, 60, 50, 40, 30, 20, 10, or 5 mg copper per kg protein isolate. In some embodiments, the protein isolate has a copper level has a copper level from 1 to 100, from 1 to 5, from 5 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 1 to 20, from 1 to 10, from 5 to 90, from 5 to 80, from 5 to 70, from 5 to 60, from 5 to 50, from 5 to 40, from 5 to 30, from 5 to 20, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, from 20 to 40 mg copper/kg protein isolate.

Protein isolate prepared according to the present disclosure may be used in preparing various animal feed or human food, such as used as meat analogues in, for example, hamburgers and sausage, in dairy free ice cream or yogurt, frozen desserts, protein powders, protein fortified food or meals (e.g., protein fortified snacks, such as chips and crackers), nutritional beverages, baked good (e.g., cakes, cookies, brownie, bread), protein bars, protein pudding or protein gels, dressing, dips, mayonanaise, coffee creamer, creamy sauces or soups, and meringue. Additional description of potential use of protein isolate may be found at the U.S. provisional application titled “Food Compositions Comprising Methylococcus Capsulatus Protein Isolate” filed on Oct. 7, 2019.

In certain embodiments, the protein isolate is processed from bacterial biomass under GMP conditions.

### EXAMPLES

### Example 1

### Growth of Methylococcus Capsulatus Bath with Varying Copper Concentration in a Continuous Culture System

The wild-type M. capsulatus Bath was grown in continuous fermentation in 2 L vessels. Nutrients required for growth, except varying amounts of copper, were provided in excess with nutrient master mix feed (MMF). The composition of MMF is shown in Table 1.

TABLE 1
Composition of Master Mix Feed

| | | Concen- | | | |
|---|---|---|---|---|---|
| Material | Source | tration | Units | Material | Units |
| H₃PO₄ | Stock | 85 | % (w/w) | 0.948 | g |
| MgSO₄•7H₂O | Salt | 100 | % | 0.456 | g |
| K₂SO₄ | Salt | 100 | % | 0.201 | g |
| FeSO₄•7H₂O | Salt | 100 | % | 0.025 | g |
| ZnSO₄•7H₂O | Sol-n | 6 | g/L | 0.0264 | mL |
| MnSO₄•H₂O | Sol-n | 2 | g/L | 0.0051 | mL |
| CoSO₄•7H₂O | Sol-n | 2 | g/L | 0.0114 | mL |
| Na₂MoO₄•2H₂O | Sol-n | 2 | g/L | 0.0201 | mL |
| NiCl₂•6H₂O | Sol-n | 2 | g/L | 0.0055 | mL |
| DI Water QS | | | | 1000 | mL |

M. capsulatus Bath has ability to uptake copper into the cell, then use or store all provided copper in concentrations within ranges tested in Example 2 below. To determine the impact of copper concentration on crude protein, copper was fed by a syringe pump at calculated feed rates. The calculation was based on the assumption, that all copper fed, was consumed by the bacteria. For example: for a low copper (Cu) concentration of 50 μg Cu/g of DCW (dry cell weight) and harvest rate 5 g/L/h of DCW, Cu—CuSO₄.5H₂O feed should be 250 μg/L/h.

Conditions for the methane continuous fermentation are provided in Table 2.

TABLE 2
Parameters for continuous culture with methane

| Parameter | Conditions/Comment | |
|---|---|---|
| Working volume | 1.5 | L |
| Temperature | 42° | C. |
| Agitation | 1200 | RPM |
| Micro-sparger, (20 μM) | Methane | |
| Methane flow | 100 | mL/min |
| Ring sparger | Air | |
| Air Flow to control pO₂ | 360-720 | mL/min |
| pO₂ set point | 10% by Air flow | |
| pH set point | 6.5 | |
| pH control | 1N NaOH, 0.5M H₂SO₄ | |
| Master Mix Feed (MMF) | No copper addition | |
| MMF Power | Supports growth up to 15 g/L of DCW | |
| Nitrogen feed | 0.5M HNO₃ | |
| N-NO₃ range | 5-60 | mg/L |
| Dilution Rate | 0.1 | 1/h |

### Biomass Collection

Throughout the experiment, wash-out periods were applied after change of condition to wash out biomass obtained at the previous copper concentration and establish new steady state fermentation. Length of wash out period was 20-24 hours or two fermentation volumes. For each set of conditions, two to three liters of the continuously pumped out fermentation broth were collected, which was a volume to obtain 15-20 grams of dry cell weight biomass. During collection the fermentation broth was stored in fridge. The collected broth was centrifuged and the wet cell pellets were stored at −80° C. Next, pellets were lyophilized, and the dry cell biomass was subjected to crude protein and elemental analysis. Gas analysis of methane, oxygen, and carbon dioxide was performed during biomass collection periods.

### Example 2

### Protein Isolation Using Microfiltration and Ultrafiltration

### Methods and Materials

Fermentation broth was collected from continuous fermentation and centrifuged. The liquid was discarded. Collected biomass was resuspended in cold de-ionized water to 6-8% total solids. The pH of the mixture adjusted to pH 8 using 5N sodium hydroxide. The solution was homogenized using a Microfluidics LM-10 processor set at 22,000 psi or 1300 bar. The solution was homogenized using 1 pass through the processor and kept cool on ice. The pH was readjusted after homogenization from pH 7 to pH 8 using 5N sodium hydroxide.

The homogenized solution was then adjusted to a total solids of 2% with cold water and centrifuged at 3000×g for 5 minutes at 10° C. The supernatant was carefully decanted into a clean beaker and the pellet discarded. The supernatant solution was subjected to microfiltration using a Millepore Pellicon 2 system and a Durapore 0.65 um filter cassette. The retentate pressure was maintained at 5 psi. The retentate volume was maintained constant with the addition of water. 2-3 volumes of permeate were collected. The permeate was concentrated 10× via ultrafiltration using a Pellicon 2 10 kDa filter cassette. The retentate was freeze dried using a Columbia International Vacuum Freeze Dryer model FD50-B2A.

The dried protein isolate was characterized by % crude protein using the Dumas method using a LECO 828 nitrogen analyzer. Briefly, a measured sample of dry protein isolate powder was rapidly combusted in a hot furnace under a pure oxygen environment. Released nitrogen in the combustion gas was measured by a thermal conductivity detector using helium as the carrier gas. Moisture in the combustion gas was removed by a thermoelectric cooler. The nitrogen content of the protein isolate sample was determined by comparing the amount of released nitrogen to calibration standards with known nitrogen content. The crude protein content in the protein isolate was calculated by multiplying the nitrogen content of the sample by a nitrogen to protein conversion factor.

The nucleic acid content was determined using a Lucigen Masterpure Complete DNA & RNA Purification Kit MC85200.

A flow chart for the protein isolation process using microfiltration followed with ultrafiltration is shown in FIG. 1.

### Results

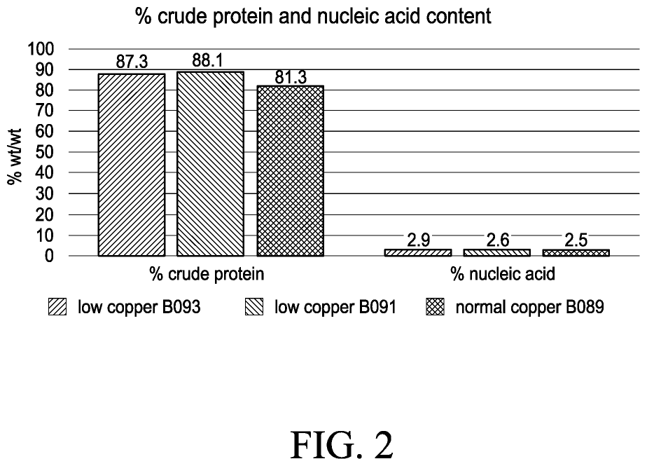

Protein isolate produced from biomass grown under low copper conditions (25 mg copper/kg biomass) was shown to have a higher percentage crude protein of 87-88% compared to protein isolate produced from biomass grown with normal copper levels (154 mg/kg) (see FIG. 2). The nucleic acid content remained low at 2-3% of the total weight of the protein isolate under both low and normal copper conditions.

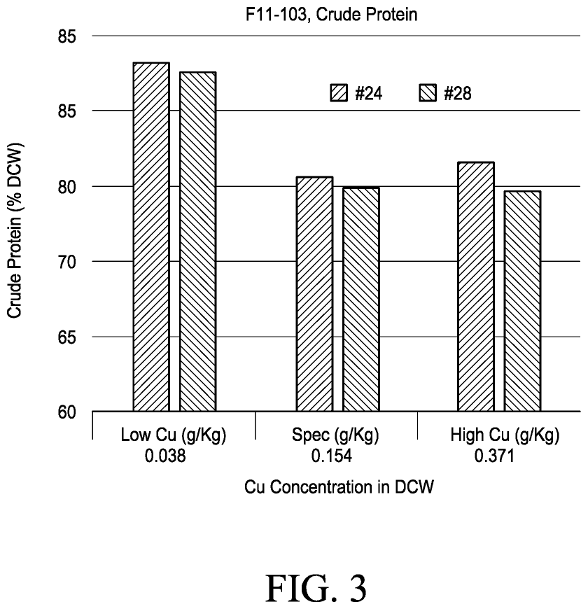

FIG. 3 shows the increase in crude protein in the biomass collected from fermentations run under a reduced amount of copper. Fermentations under low copper (0.038 g/kg) produced crude protein of 82-83% while fermentations under normal (0.154 g/kg) and high copper (0.371 g/kg) levels produced crude protein of about 75% and 75-77%, respectively.

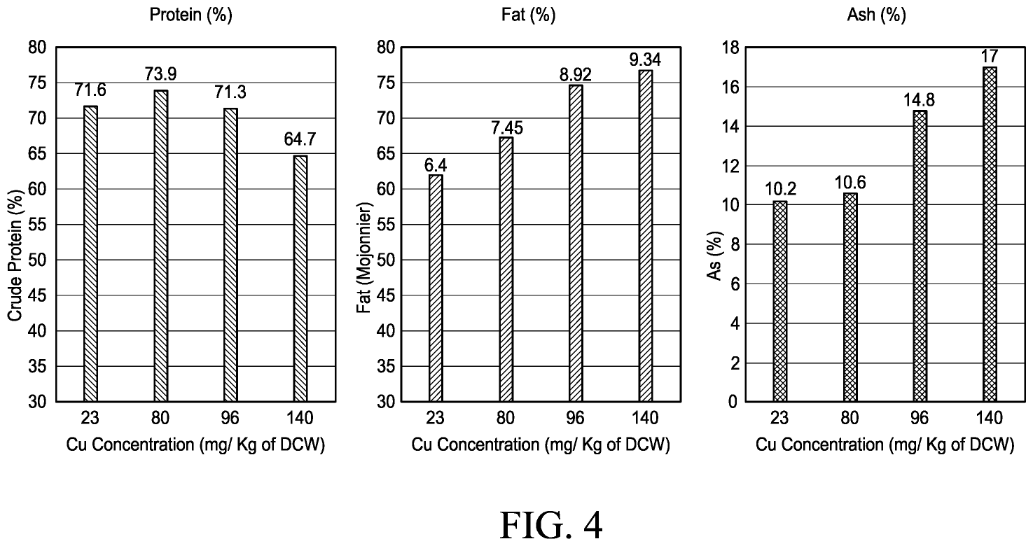

FIG. 4 shows the results of the analysis of crude protein, fat and ash content of the biomass grown under those variable copper conditions (23, 80, 96 and 140 mg/kg). Reduced copper levels increased crude protein percentages of protein isolates while reduced fat and ash percentages.

In summary, the results show higher crude protein percentages from low copper grown biomass compared to normal copper grown biomass (FIGS. 3 and 4). In addition, the higher crude protein of the biomass was maintained in the protein isolate product under the same downstream processing (DSP) conditions (FIG. 2).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/911,747, filed Oct. 7, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for generating biomass, comprising:
(a) continuously culturing a methanotrophic bacterium at a copper level no more than 100 mg copper per kg dry cell weight (DCW) to generate biomass.

2. The method of claim 1, wherein the methanotrophic bacterium is Methylococcous capsulatus Bath.

3. The method of claim 1 or claim 2, wherein the copper level is between 20 and 70 mg copper per kg DCW.

4. The method of any of claims 1 to 3, wherein the biomass comprises at least 71% crude protein, preferably at least 80% crude protein.

5. The method of any of claims 1 to 4, wherein the biomass comprises at most 7.5% fat.

6. The method of any of claims 1 to 5, wherein the biomass comprises at most 11% ash.

7. The method of any of claims 1 to 6, wherein the biomass comprises at most 10% nucleic acid, preferably at most 5% nucleic acid.

8. The method of any one of claims 1 to 7, further comprising:
(b) purifying proteins from the biomass to generate protein isolate.

9. The method of claim 8, wherein step (b) comprises (i) disrupting cells of the biomass to generate a lysate, and (ii) separating and/or concentrating proteins from the lysate.

10. The method of claim 8 or claim 9, wherein the protein isolate has at least 82% crude protein, preferably at least 85% crude protein.

11. The method of any of claims 8 to 10, wherein the yield of the protein isolate is more than the yield of a protein isolate prepared in the same manner except that the methanotrophic bacterium is cultured at a copper level of 150 mg copper per kg DCW.

12. The method of any of claims 8 to 11, wherein the yield of the protein isolate is at least about 15%, preferably at least about 20%.

13. The method of any of claims 8 to 12, wherein the ratio of the yield of the protein isolate to the yield of the protein isolate prepared in the same manner except that the methanotrophic bacterium is cultured at the copper level of 150 mg copper per kg DCW is at least 1.2 or at least 1.5, preferably at least 2.0 or at least 2.5.

14. The method of any of claims 8 to 13, wherein the protein isolate comprises at most 3% nucleic acid.

15. A bacterial biomass comprising primarily, consisting essentially of, or consisting of, a biomass of a methanotrophic bacterium having a copper level no more than 100 mg copper per kg dry cell weight (DCW).

16. The bacterial biomass of claim 15, wherein the bacterial biomass and/or the biomass of the methanotrophic bacterium has a copper level in the range of 20 to 70 mg copper per kg DCW.

17. The bacterial biomass of claim 15 or claim 16, wherein the bacterial biomass and/or the biomass of the methanotrophic bacterium has at least 71% crude protein, preferably at least 80% crude protein.

18. The bacterial biomass of any of claims 15 to 17, wherein the bacterial biomass and/or the biomass of the methanotrophic bacterium has at most 7.5% fat.

19. The biomass of any of claims 15 to 18, wherein the bacterial biomass and/or the biomass of the methanotrophic bacterium comprises at most 11% ash.

20. The bacterial biomass of any of claims 15 to 19, wherein the bacterial biomass and/or the biomass of the methanotrophic bacterium comprises at most 10% nucleic acid, preferably at most 5% nucleic acid.

21. The bacterial biomass of any of claims 15 to 20, wherein the methanotrophic bacterium is Methylococcous capsulatus Bath.

22. A protein isolate prepared from a bacterial biomass that comprises primarily a biomass of a methanotrophic bacterium, wherein the protein isolate comprises at least 82% crude protein, preferably at least 85% crude protein.

23. The protein isolate of claim 22, wherein the protein isolate comprise at most 3% nucleic acid.

24. The protein isolate of claim 22 or claim 23, wherein the methanotrophic bacterium is Methylococcous capsulatus Bath.

25. The protein isolate of any of claims 22 to 24, wherein the bacterial biomass and/or the biomass of the methanotrophic bacteria has a copper level no more than 100 mg copper per kg dry cell weight (DCW).

26. The protein isolate of any of claims 22 to 25, wherein the protein isolate has a copper level no more than 100 mg copper per kg protein isolate.
